(19) United States
(12) Patent Application Publication
(10) Pub. No.: US 2025/0381047 A1
(43) Pub. Date: Dec. 18, 2025

(54) METHOD FOR SETTING UP AN ORTHOPAEDIC DEVICE

(71) Applicant: Ottobock SE & Co. KGaA, Duderstadt (DE)

(72) Inventors: Christian Daur, Berlin (DE); Andreas Leiniger, Leinefelde OT Birkungen (DE); Christian Käppel, Berlin (DE); Lüder Mosler, Duderstadt (DE)

(21) Appl. No.: 18/862,382

(22) PCT Filed: May 5, 2023
(86) PCT No.: PCT/EP2023/061964

§ 371 Date: Nov. 1, 2024

(30) Foreign Application Priority Data
May 6, 2022 (DE) 10 2022 111 327.2

(51) Int. Cl. A61F 2/76; A61F 2/50

(52) U.S. Cl. CPC A61F 2/76; A61F 2/5046; A61F 2002/7695

(57) ABSTRACT

The invention relates to a method for the computer-based setting up of an orthopaedic device which is worn on the body of a patient provided therewith, the method comprising the following computer-implemented steps:—providing a digital model of the body:—providing body-related patient parameters for fitting the patient with an orthopaedic device; —providing a digital orthopaedic care proposal which comprises providing the patient with an orthopaedic care model and with care parameters associated therewith:—using a simulation device to simulate orthopaedic care for the patient on the model of the body with the orthopaedic care proposal, taking account of the body-related patient parameters provided,—wherein, on the basis of the simulation, an interaction between the model of the body and the orthopaedic care proposal is ascertained by means of an evaluation unit; and—determining on the basis of the orthopaedic care proposal already provided an optimized orthopaedic care proposal in dependence on the interaction ascertained by means of the evaluation unit.

The invention relates to a method of computer-based configuration of an orthopedic device worn on the body of a patient equipped therewith. The invention also relates to a computer program to this end.

Within the meaning of the present invention, orthopedic devices are in particular orthoses, prostheses, exoskeletons and optionally also wheelchairs, as well as the associated seat shells and seat cushions that are adapted to the user on an individual basis. Orthoses are products that support, assist, protect or restrict the freedom of movement of a body part of the patient, for example a joint, in order to avoid overuse. By contrast, prostheses replace body parts of the patient that are not, or no longer, present. Exoskeletons are mechanical support structures in particular, which are in-tended to support, assist or protect the main musculoskeletal system of the patient.

Hereinbelow, a patient is understood to mean any user of the orthopedic device. This consequently relates to the wearer of the orthopedic device.

As a rule, an orthopedic device is arranged on a body part of the patient. There need not necessarily be contact with the skin of the patient. For example, orthoses and exoskeletons are frequently worn above the apparel, and so the latter, for example a pair of trousers, is situated between the orthopedic device and the skin of the patient. Nevertheless, a knee orthosis is secured to the knee or the leg of the patient, for example. A prosthesis always comprises an interface element that is connected to an amputation stump or any other body part and secured to the respective body part. In the case of a leg prosthesis, use is made of a prosthesis socket, for example, which represents the interface between prosthesis and amputation stump. In this case, the amputation stump would be the body part of the patient. As a rule, a liner is used between the skin surface of the amputation stump of the patient and the prostheses socket in order to reduce shearing forces that act on the skin.

As a rule, a prosthesis socket for an amputation stump is produced from rigid (hardly deformable) material, for example a fiber-reinforced plastic, and forms an important part of the interface between the amputation stump and the prosthesis arranged on the prosthesis socket. Appropriate prosthesis sockets have been used for a long time, especially for leg prostheses that should be arranged on an amputation stump, for example a thigh.

Prosthesis sockets for leg amputees, in particular, are exposed to particular loads during daily use. When walking, the entire weight of the patient bears on the prosthesis socket, and hence in particular on the amputation stump arranged in the prosthesis socket. It is therefore very important to adapt the prosthesis socket as optimally as possible to the individual circumstances and needs of the patient, in particular to the shape and geometry of the relevant body part, wherein the different movement situations in particular, such as walking, standing or sitting down, should be taken into consideration.

The patient is assisted by a specialist, usually trained in the art, for example an orthopedic technician, when fitting and configuring an orthopedic device. The orthopedic technician can draw on their knowledge and experience to implement the necessary adjustments so that the orthopedic device fits optimally, and a sparing movement pattern arises.

EP 2 153 370 B1 has disclosed a system for aligning prostheses, in which both movement data of the person equipped with the prosthesis and prosthesis alignment errors are ascertained from a movement database. These are compared with one another in order to determine whether the prosthesis corresponds to a target value or requires further adjustments.

DE 10 2012 009 507 A1 has disclosed a method and an apparatus for determining incorrect positions in the construction of prostheses of the lower extremities, wherein inertial measurement data are ascertained over at least one gait cycle and are compared to target values.

DE 10 2018 128 514 B4 has disclosed a method for carrying out static prostheses construction for a prosthesis, wherein a plurality of components are arranged on one another. In this case, an actual position and an actual orientation of the components arranged on one another are ascertained relative to one another on the basis of detected positions and orientations of markers and are compared to appropriate target values. In so doing, it is only the components that are compared among themselves without consideration being given to the individual situation of the patient.

Within the scope of configuring an orthopedic device, which comprises the construction and the selection of an orthopedic device, the adaptation of an available orthopedic device and the setting of parameters of the orthopedic device, it is necessary to ensure that the orthopedic device is configured in such a way that this does not result in any adverse effects on the remaining body parts or on the entire body. Furthermore, the configuration can also contain the automatic adaptation and/or creation of individualized production data for the orthopedic device, for example for subsequent additive manufacturing.

Against this background, the problem addressed by the present invention is that of proposing an improved method for configuring an orthopedic apparatus.

According to the invention, the problem is solved by the method of configuration of an orthopedic device as claimed in claim 1. Advantageous embodiments of the invention are found in the corresponding dependent claims.

As claimed in claim 1, a method of computer-based configuration of an orthopedic device worn on the body of a patient equipped therewith is proposed, wherein the method comprises the following steps which are or can be executed by a data processing system:
providing a digital body model;
providing body-related patient parameters for treatment of the patient with an orthopedic device;
providing a digital orthopedic treatment proposal which comprises the treatment of the patient with an orthopedic treatment model and treatment parameters related thereto;
using a simulation device to simulate an orthopedic treatment of the patient on the body model using the orthopedic treatment proposal, with consideration being given to the body-related patient parameters provided,
wherein an interaction between the body model and the orthopedic treatment proposal is ascertained on the basis of the simulation by means of an evaluation unit; and
using the evaluation unit to determine an optimized orthopedic treatment proposal depending on the ascertained interaction using the previously provided orthopedic treatment proposal as a basis.

Accordingly, the invention proposes that an orthopedic treatment of the patient is simulated on the digital body model provided using the orthopedic treatment proposal provided, wherein the body-related patient parameters provided are taken into account in the simulation of the orthopedic treatment proposal. In this case, the simulation ascertains an interaction between the body model and the treatment proposal and can thus ascertain the effects of the provided treatment proposal on the digital body model. As a result, the effects that the treatment proposal with the proposed setting parameters might have or will have on the patient can already be determined before the patient is treated with the treatment proposal.

Now, using the ascertained interaction between the digital body model and the digital treatment proposal as a basis, the evaluation unit of the data processing system is used to ascertain an optimized orthopedic treatment proposal. In so doing, the optimized orthopedic treatment proposal is ascertained for example by adapting/modifying the original treatment proposal provided. In this case, the optimized orthopedic treatment proposal contains measures for configuring the physical orthopedic device in particular, to the effect that the treatment target is achieved to the best possible extent and, additionally, negative effects on the body of the patient are lessened, reduced or entirely avoided.

Once an optimized orthopedic treatment proposal has been determined by the evaluation unit, it can be displayed on a display of the data processing system. In the process, the specialist can optionally start a further simulation iteration until an orthopedic treatment proposal was obtained which, in the view of the specialist, is optimized to the greatest possible extent.

Now, the orthopedic device of the patient is configured on the basis of an optimized orthopedic treatment proposal ascertained by the method so that the orthopedic device configured thus can be worn on the body of the patient. Accordingly, the orthopedic device of the patient is configured as proposed by the optimized orthopedic treatment proposal from the simulation.

For the first time, this makes it possible to ascertain negative effects on the body of the patient when configuring an orthopedic device without the patient, and so an optimally configured orthopedic device can be fitted to the patient.

In this context, the digital body model provided can be a generic body model that is individualized by the body-related patient parameters. In particular, the body model can be a digital (standardized) anatomical body model.

According to an embodiment, provision is made for the optimized orthopedic treatment proposal to be used as a basis for a renewed simulation of the orthopedic treatment.

The optimized treatment proposal found by the simulation is thus used as a basis for a renewed simulation by virtue of the measures for configuring the orthopedic device contained in the found optimized treatment proposal being applied to the previously provided treatment proposal, on which the simulation was based. As a result, the simulation can iteratively approach (globally or locally) an optimum and supply a best-possible result.

According to an embodiment, provision is made for the orthopedic treatment proposal provided to be created automatically in advance by means of a computing unit on the basis of at least some of the body-related patient parameters.

The computing unit can automatically create an orthopedic treatment proposal on the basis of the patient parameters, which can also contain pathological information, for example on the basis of the body-related information about the patient provided to the computing unit in advance. For example, a treatment proposal for an appropriate prosthesis can thus be created with knowledge of corresponding amputation information, said treatment proposal being adapted to the body-related patient parameters and the information contained therein.

In this case, appropriate preset treatment proposals for various combinations of patient parameters can be stored in a database or a machine learning system. Now, one of this multiplicity of preset treatment proposals is selected on the basis of specific patient parameters, and the simulation is based thereon. In this case, provision can likewise be made for the computing unit to appropriately adapt the preset treatment proposal on the basis of the patient parameters before the simulation is carried out.

According to one embodiment, provision is made for the digital body model to model at least a portion of the human skeletal system, if need be also at least one joint function in this respect.

In this case, provision can be made for a simulation of the muscles, tendons and ligaments, and optionally also the control thereof by the nerves, to also be carried out for the purpose of simulating the joint function; this can be relevant on account of medical limitations in particular.

According to one embodiment, provision is made for the digital body model to be adapted to the patient on the basis of at least some of the body-related patient parameters and/or for the digital body model to be individualized on the basis of measured data from a measurement of the physical patient body performed in advance.

In this case, provision can be made for the digital body model to be provided as a generic body model and be adapted to the peculiarities of the patient by way of the body-related patient parameters. However, the digital body model can also be individualized individually on the basis of measured data from a measurement carried out in advance, wherein the simulation can be based on an even better likeness of the patient. For example, the measurement in this context may consist of recording individual measurements of the patient or else a partial or complete 3-D scan. By preference, as much information as possible is provided, even from areas away from the region to be treated, for example information about body parts that do not directly affect the treatment proposal (e.g. adjacent joints).

According to one embodiment, provision is made for determination of an optimized orthopedic treatment proposal to comprise modification of the treatment parameters of the orthopedic treatment model and/or selection of the orthopedic treatment model.

Accordingly, the treatment proposal can contain measures which modify the treatment proposal on which the simulation is based, in such a way that in particular negative interactions in certain body parts or the entire body of the patient in are lessened, reduced or entirely avoided, while at the same time the treatment target is attained to the best possible extent. For example, such measures can represent setting specific parameters with which the orthopedic device can be set. In the case of (microprocessor-controlled) prostheses, this for example relates to specific damping properties, angle positions or similar measures. It is usually the case that the number of modifiable parameters increases as the prosthesis becomes more complex; in the case of microprocessor-controlled prostheses in particular, this modification is also performable in automated fashion by the software.

However, the optimized treatment proposal can also contain measures that comprise the selection of a specific orthopedic treatment model. Thus, the simulated interactions and the effect on the body of the patient arising therefrom can render necessary a change of the orthopedic treatment model on which the treatment proposal is based and a replacement by another treatment model, for example a different type of treatment model.

According to one embodiment, provision is made for the scope of the simulation to include simulation of a static load case with the simulated orthopedic treatment, wherein sites of pressure, points of contact, areas of contact and/or deviations of the overall structure from a specified structure of the orthopedic treatment are ascertained as interaction between the body model and the orthopedic treatment proposal.

In a static load case, a load on the orthopedic device is simulated, in the case of which the orthopedic device is not moved and instead loaded by a static force. This is the case for standing, sitting and lying, in particular. The simulation of the static load case allows ascertainment of static-type interactions between the body model and the treatment proposal, for example sites of pressure and points of contact, which might cause the patient pain. On the basis thereof, it is subsequently possible to create an optimized treatment proposal which for example provides measures for moderating these sites of pressure and points of contact and thus increasing the patient comfort or else rectifying existing joint malpositions. In this respect, this is also necessary as this avoids protective postures.

According to one embodiment, provision is made for the scope of the simulation to include simulation of a dynamic load case with the simulated orthopedic treatment, in which the orthopedic treatment is used to simulate a specific movement or a specific movement sequence, wherein a load on the affected joints, a scope of movement and/or a deviation between the simulated movement and a specified optimal movement or between the simulated movement sequence and a specified optimal movement sequence are ascertained as interaction between the body model and the orthopedic treatment proposal.

In the dynamic load case, a movement or a movement sequence is simulated, and the interaction between the body model and the treatment proposal during the movement is ascertained in the process. In particular, this relates to the load on the affected joints, the scope of movements and deviations from an optimal movement sequence. On the basis of the interaction ascertained thus and the negative effects on the body of the patient, it is subsequently possible to create a corresponding optimized treatment proposal containing measures that lessens or prevents these negative effects.

According to one embodiment, provision is made for an effect on at least one joint of a static or dynamic load case for the simulated orthopedic treatment to be ascertained as interaction between the body model and the orthopedic treatment proposal, the joint differing from the directly treated joint and/or being provided within a load case-related joint chain of the body model.

Thus, the interaction between the body model and the treatment proposal does not only take account of the directly treated joint; instead, consideration is also given to those joints that differ therefrom or are provided within a load case-related joint chain.

For example, when simulating a knee orthosis, it can thus be necessary to also consider the effects on the hip joint or the spinal column if the intention is to create an optimized treatment proposal. In this case, joints of a joint chain are those joints which, using the treated joint or the replaced joint as a starting point, are decisively involved in and required for the movement sequence.

In addition to the load-related interactions between orthopedic treatment and body model, it is also possible to include further interactions, for example the controllability of the treatment by the remaining muscular strength. In the case of EMG-controlled prostheses or orthoses in particular, it is possible to consider the interaction between muscular signals of the model and the sensor system of the treatment in order thus to optimally align the sensor system.

According to one embodiment, provision is made for the optimized orthopedic treatment proposal to comprise at least one measure on a body part and/or joint that differs from the directly treated one.

The optimized orthopedic treatment proposal can accordingly also contain measures that relate to other body parts or joints in order to lessen or prevent the negative effects on the body of the patient that arise from the simulated interaction between the body model and the treatment proposal.

In a further embodiment, it is conceivable that there is a comparison between a generic body model without parameterization by the patient parameters and a generic body model with parameterization by the patient parameters. In the process, a simulation containing the provided orthopedic treatment proposal in relation to the generic body model without parameterization and a simulation containing the provided orthopedic treatment proposal in relation to the generic body model with parameterization are carried out, wherein the two simulations are compared and the comparison is then used to ascertain the interaction in order to thus determine an optimized orthopedic treatment proposal.

With reference to the attached figures, the invention is explained in exemplary fashion. In the drawing:

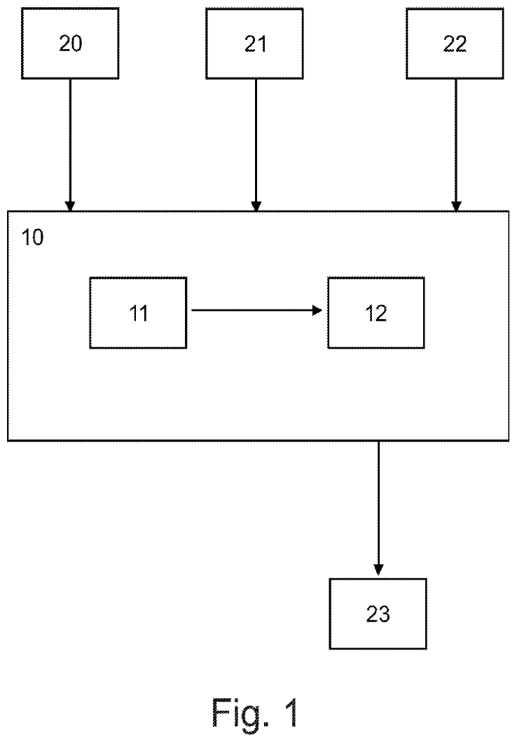

FIG. 1 shows a schematic illustration of the method in relation to the implementing data processing system;

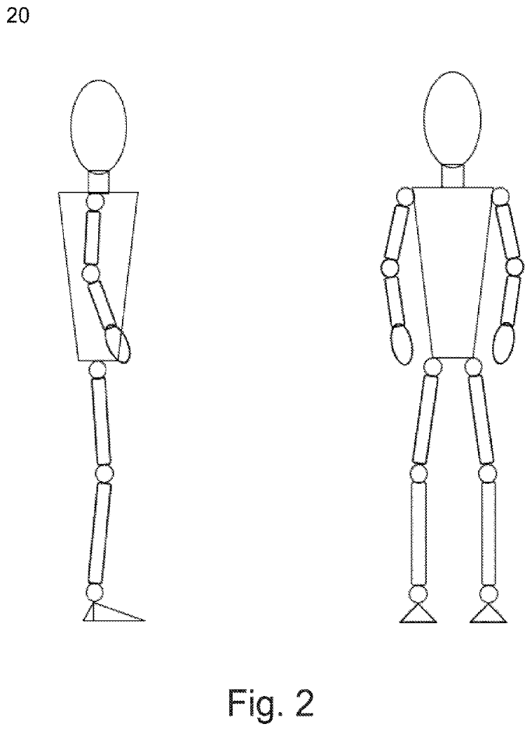

FIG. 2 shows a schematic illustration of a generic body model in the form of an avatar;

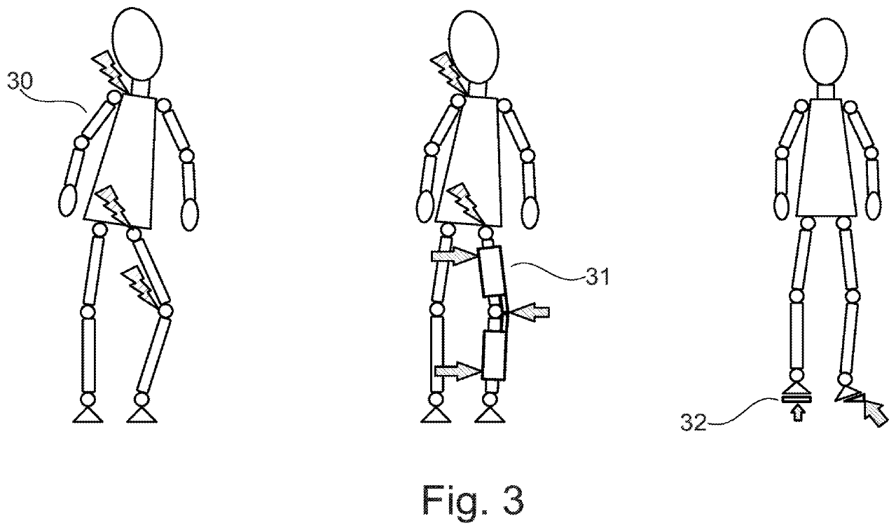

FIG. 3 shows a schematic illustration of a static simulation in a first embodiment;

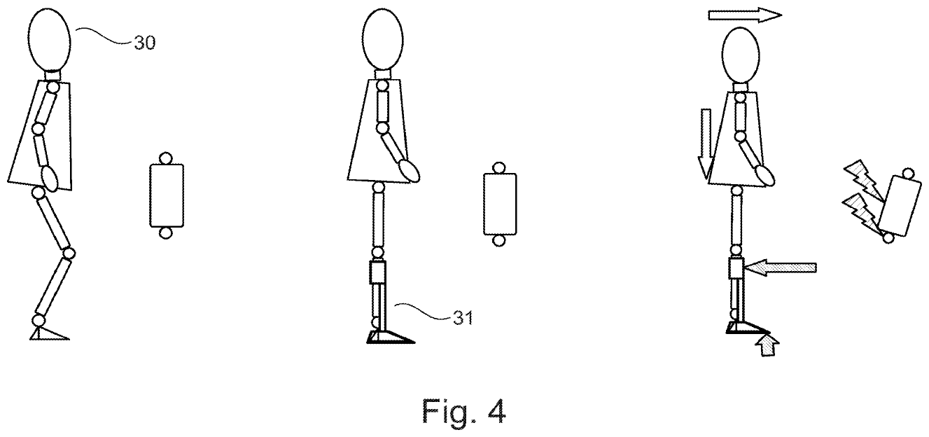

FIG. 4 shows a schematic illustration of a static simulation in a second embodiment; and

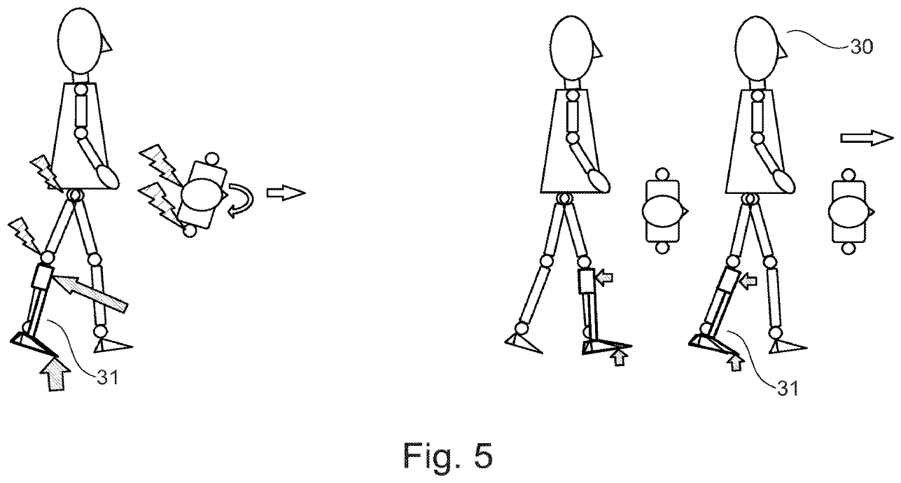

FIG. 5 shows a schematic illustration of a dynamic simulation.

In a schematically much simplified illustration, FIG. 1 shows a data processing system 10 which has a simulation device 11 and an evaluation unit 12. In this case, the simulation device 11 and the evaluation unit 12 can represent software modules that are executed in the data processing system 10 and interact with one another in the process in order to be able to carry out the method described above.

Now, a digital body model 20, which can be a generic body model for example, is initially provided to the data processing system 10. Moreover, the data processing system 10 is provided with appropriate body-related patient parameters 21, which relate to corresponding body-related peculiarities of the patient. In the process, the generic digital body model 20 can be augmented with the aid of the body-related patient parameters 21 provided, to the extent that the body model in conjunction with the patient parameters models the body of the patient.

Finally, a digital orthopedic treatment proposal 22, which should be simulated in conjunction with the body model and the patient parameters, is provided to the data processing system 10.

In this case, the body model 20, the patient parameters 21 and the treatment proposal 22 can also be provided to the data processing system 10 by way of a database, in which the individual data are stored.

Now, the provided orthopedic treatment proposal 22 is simulated with the aid of the simulation device 11 on the body model 20 with the parameterized patient parameters 21, wherein both a static and a dynamic load case can be simulated in the process. On the basis of this simulation, the evaluation unit 12 then ascertains an interaction between the body model and the treatment proposal, in order to be able to subsequently determine an optimized orthopedic treatment proposal 23 therefrom.

In a schematic illustration, FIG. 2 shows a digital body model 20 which is depicted in a side view on the left-hand side and in a front view on the right-hand side. The digital body model 20 in FIG. 2 is depicted in the form of a generic body model and, in particular, contains the joints and body parts required for movement. In this case, the digital body model 20 is implemented in such a way that it contains appropriate re-strictions for the movement of each joint, in order to thus model the movement of a human.

FIG. 3 shows an exemplary embodiment as to how an orthopedic treatment proposal is ascertained on the basis of the simulation. In this case, the body model 30 is shown on the left-hand side; it is parameterized appropriately by way of the patient parameters.

In this case, the patient suffers from both a left bow leg and a leg length discrepancy. Here, these patient data are supplied in the form of patient parameters to the generic body model 20 in order to create therefrom the patient-related body model 30 visible in FIG. 3 (left-hand illustration). In this context, it is evident that corresponding discomfort in the left knee, in the hip joint on the left-hand side here and in the region of the cervical vertebrae arises in a simulation.

The central view shows the body model 30 which is equipped with a treatment proposal 31 for treating the bow leg. This combination of patient-related body model 30 and the treatment proposal 31 equipped therewith is now simulated with the aid of the static simulation in order to ascertain the interaction of this treatment proposal 31 with the remaining body of the patient. In the central view, this is characterized by the fact that patient discomfort will probably still exist in the left hip joint and in the region of the cervical vertebrae, even with the proposed treatment proposal 31.

By way of the simulation and the evaluation of the simulation accompanying this, the evaluation unit then determines that the treatment proposal 31 rectified the discomfort in the left knee but did not rectify the discomfort in the left hip joint and in the region of the cervical vertebrae that arises from the leg length discrepancy.

The optimized orthopedic treatment proposal arising therefrom therefore provides for measures to compensate for the leg length discrepancy. The optimized treatment proposal therefore provides for the use of inserts 32 to reduce the discomfort of the patient.

Therefore, the optimized orthopedic treatment proposal provides for the knee orthosis 31 to be replaced by inserts 32 in order to address both the malposition of the left knee due to the bow leg and the discomfort in the left hip joint and in the region of the cervical vertebrae.

FIG. 4 shows an exemplary embodiment in which, on the left-hand side, the body model 30 was once again parameterized with the patient parameters. In the central view, the patient was simulated with a treatment proposal 31 for the body model, wherein the patient can be seen once in the side view and once in the top view. The simulation shows that although the static load cases are without findings, the dynamic load case includes a compensation movement of the hip and the spinal column. In the case illustrated here, emulating the toe push-off by the orthosis leads to a hyperextension of the knee. The hip on the affected side is rotated backwards as a compensation movement. This furthermore leads to an unwanted rotation in the spinal column and possible long-term damage to the intervertebral bodies. This cannot, or can only hardly, be identified in the case of a local consideration of the affected region and would thus lead to a treatment that is disadvantageous for the patient.

The optimized orthopedic treatment proposal can provide for the treatment proposal 31 to be set accordingly such that the existing compensation movement of the hip and the spinal column no longer occurs. To this end, it is possible to set appropriate parameters for the simulated orthopedic device, which then reduces the discomfort. In the present example, a minor flexion of the lower leg could be set; this reduces the corrective effect of the orthosis and avoids the overcorrection. Alternatively, a heel of the orthosis could be raised. A further alternative would lie in the use of a softer or shorter footplate, whereby the front lever arm would be shortened, and the dynamics would be influenced positively. The system can compare the various alternatives and also check these in further rounds of simulation. For example, this could give rise to the determination that the heel height adjustment further improves the static case but supplies no solution to the problems in the dynamic case and should therefore be dis-carded.

FIG. 5 shows an exemplary embodiment in which a dynamic load case is simulated for a parameterized body model 30 with a treatment proposal 31. It is evident from the left-hand view that the provided treatment proposal 31 and the set parameters thereof overcorrect, whereby discomfort arises in the knee and also in the hip joint and the spinal column.

An optimized treatment proposal 31 can be identified on the right-hand side; it no longer exhibits negative interactions in the dynamic load case. In this case, the optimized orthopedic treatment proposal can contain measures that suggest a correction of the set parameters of the treatment proposal 31 in order to reduce the overcorrection.

### LIST OF REFERENCE SIGNS

10 Data processing system
11 Simulation device
12 Evaluation unit
20 Digital body model
21 Patient parameter
22 Provided digital orthopedic treatment proposal
23 Optimized orthopedic treatment proposal
30 Parameterized body model
31 Digital treatment proposal

1. A computer-implemented method performed in an orthopedic device worn on the body of a patient equipped therewith, wherein the method comprises the following computer-implemented steps:
providing a digital body model;
providing body-related patient parameters for treatment of the patient with an orthopedic device;
providing a digital orthopedic treatment proposal which comprises the treatment of the patient with an orthopedic treatment model and treatment parameters related thereto;
using a simulation device to simulate an orthopedic treatment of the patient on the body model using the orthopedic treatment proposal, with consideration being given to the body-related patient parameters provided,
wherein an interaction between the body model and the orthopedic treatment proposal is ascertained on the basis of the simulation by means of an evaluation unit; and
using the evaluation unit to determine an optimized orthopedic treatment proposal depending on the ascertained interaction using the already provided orthopedic treatment proposal as a basis.

2. The method of claim 1, wherein the optimized orthopedic treatment proposal is used as a basis for a renewed simulation of the orthopedic treatment.

3. The method of claim 1, wherein the orthopedic treatment proposal provided is created automatically in advance by a computing unit on the basis of at least some of the body-related patient parameters.

4. The method of claim 1, wherein the digital body model models at least a portion of the human skeletal system.

5. The method of claim 1, wherein the digital body model is or has been adapted to the patient on the basis of at least some of the body-related patient parameters and/or in that the digital body model is or has been individualized on the basis of measured data from a measurement of the physical patient body performed in advance.

6. The method of claim 1, wherein determination of an optimized orthopedic treatment proposal comprises modification of the treatment parameters of the orthopedic treatment model and/or selection of the orthopedic treatment model.

7. The method of claim 1, wherein the scope of the simulation includes simulation of a static load case with the simulated orthopedic treatment, wherein sites of pressure, points of contact, areas of contact and/or deviations of the overall structure from a specified structure of the orthopedic treatment are ascertained as interaction between the body model and the orthopedic treatment proposal.

8. The method of claim 1, wherein the scope of the simulation includes simulation of a dynamic load case with the simulated orthopedic treatment, in which the orthopedic treatment is used to simulate a specific movement or a specific movement sequence, wherein a load on the affected joints, a scope of movement and/or a deviation between the simulated movement and a specified optimal movement or between the simulated movement sequence and a specified optimal movement sequence are ascertained as interaction between the body model and the orthopedic treatment proposal.

9. The method of claim 1, wherein an effect on at least one joint of a static or dynamic load case for the simulated orthopedic treatment is ascertained as interaction between the body model and the orthopedic treatment proposal, the joint differing from the directly treated joint and/or being provided within a load case-related joint chain of the body model.

10. The method of claim 1, wherein the optimized orthopedic treatment proposal comprises at least one measure on a body part and/or joint that differs from the directly treated one.

11. A non-transitory computer-readable medium storing instructions that, when executed, cause one or more processors to:
provide a digital body model;
provide body-related patient parameters for treatment of the patient with an orthopedic device;
provide a digital orthopedic treatment proposal which comprises the treatment of the patient with an orthopedic treatment model and treatment parameters related thereto;
use a simulation device to simulate an orthopedic treatment of the patient on the body model using the orthopedic treatment proposal, with consideration being given to the body-related patient parameters provided, wherein an interaction between the body model and the orthopedic treatment proposal is ascertained on the basis of the simulation by means of an evaluation unit; and
use the evaluation unit to determine an optimized orthopedic treatment proposal depending on the ascertained interaction using the already provided orthopedic treatment proposal as a basis.
